(19) United States
(12) Patent Application Publication
(10) Pub. No.: US 2025/0303169 A1
(43) Pub. Date: Oct. 2, 2025

(54) IMPLANTABLE MEDICAL SYSTEMS WITH MEDICAL LEADS WITH MULTIPLE NEURAL INTERFACES

(71) Applicant: The Alfred E. Mann Foundation for Scientific Research, Valencia, CA (US)

(72) Inventors: Brian Dearden, Pasadena, CA (US); Joseph L. Calderon, Santa Clarita, CA (US); Brian Michael Shelton, Ventura, CA (US); Neil Talbot, La Crescenta, CA (US); Anthony Ng, Santa Clarita, CA (US); Oleh Zajac, Arcadia, CA (US)

(21) Appl. No.: 19/093,157

(22) Filed: Mar. 27, 2025

Related U.S. Application Data
(60) Provisional application No. 63/572,121, filed on Mar. 29, 2024.

(51) Int. Cl. A61N 1/36; A61N 1/05

(52) U.S. Cl. CPC A61N 1/3611; A61N 1/0553; A61N 1/0556; A61N 1/36139

(57) ABSTRACT

An implantable medical system includes a medical device connected to a medical lead with several neural interfaces. The system can selectively generate stimulation signals or sense neurological signals at a selected one of several neural interfaces, such as nerve cuffs. Each neural interface has at least one working electrode that is common to all the neural interfaces. Each neural interface has at least one reference electrode that is dedicated to that interface. A neural interface that has not been selected to stimulate tissue or to sense neurological signals has its dedicated reference electrode floating and not connected to power, ground or any electronic system. Any electrodes that were used for stimulation and are about to be used for neural sensing are grounded for a time interval to discharge any residual electrical charges that may remain due to neural stimulation signals.

### CROSS-REFERENCE TO RELATED APPLICATION

This application claims priority to, and the benefit of, U.S. Provisional Application No. 63/572,121, filed on Mar. 29, 2024, the entire content of which is hereby incorporated by reference.

### BACKGROUND

### 1. Field

The present disclosure relates to stimulation devices and methods of providing stimulation.

### 2. Description of the Related Art

Electrical stimulation can be provided to one or more nerves to treat one or more medical conditions, and it can be desirable to stimulate multiple nerves to effectively treat certain medical conditions. For example, stimulating both (e.g., simultaneously) the right and left hypoglossal nerves (HGN) may be more effective at treating obstructive sleep apnea (OSA) in patients experiencing complete concentric collapse. It can also be desirable to selectively provide stimulation to certain parts of a nerve, which can be provided by an electrical lead having a plurality of independently drivable electrodes. For example, the proximal portion of an HGN nerve includes both (1) nerve fascicles that control the protrusion of the tongue and (2) nerve fascicles that control the retraction of tongue, and it can be desirable at least for purposes of treating OSA to selectively stimulate the nerve fascicles that cause tongue protrusion without stimulating those that cause retraction.

However, a stimulation device configured to selectively stimulate each of multiple different nerves could require a substantial number of electrodes. Configuring the stimulation device to independently control each of such electrodes could increase the number of components, cost, complexity, and likelihood of component failure of a driver and/or other components of the stimulation device. Or, when the driver and/or other components are limited in the number of electrodes that can be independently controlled or selected, the stimulation device may be unable to stimulate the multiple different nerves with sufficient selectivity to effectively treat a specific medical condition. It is in view of this technical background that the present disclosure is provided.

This Background section is provided only for purpose of introducing certain background information relating to the present disclosure and, thus, statements made in this Background section are not admissions of prior art.

### SUMMARY

According to an aspect, the technology relates to an implantable medical system including a medical lead including a first return conductor extending from a proximal end of the medical lead to a first return electrode at a first neural interface at a first distal end of the medical lead, a second return conductor extending from the proximal end of the medical lead to a second return electrode at a second neural interface at a second distal end of the medical lead, and a working conductor extending from the proximal end of the medical lead to first and second working electrodes respectively in the first and second neural interfaces; and a plurality of subsystems including a current source, a current sink, a signal processor, a voltage source, and a ground, wherein the signal processor has at least one input and at least one output.

In some examples, the first neural interface is configured for either neural stimulation or neural sensing; and the second neural interface configured for either neural stimulation or neural sensing.

In some examples, the implantable medical system includes a matrix switch configured to selectively couple any one of the first and second return electrodes to any one of the plurality of subsystems; and selectively couple all the first and second working electrodes to any other one of the plurality of subsystems.

In some examples, any one of the electrodes can float by not being coupled via the matrix switch to any one of the plurality of subsystems.

In some examples, the implantable medical device includes a controller configured to control the operation of the matrix switch.

In some examples, the controller is configured to select one of the first neural interface and the second neural interface for either neural stimulation or neural sensing.

In some examples, the controller is configured to select the return electrode of the other one of the first neural interface and the second neural interface to be floating.

In some examples, the controller is configured to select neural stimulation; select either the first or the second neural interface for neural stimulation; switchably couple the return electrode of the neural interface selected for neural stimulation to the ground via the matrix switch; switchably couple all the first and second working electrodes to the current source via the matrix switch; and float the return electrode of the neural interface not selected for neural stimulation.

In some examples, the controller is configured to select neural sensing after neural stimulation; select either the first or the second neural interface for neural sensing; switchably couple the return electrode of the neural interface selected for neural sensing to the ground via the matrix switch; switchably couple all the first and second working electrodes to the ground via the matrix switch for a set period of time; switchably couple all the first and second working electrodes to an input of the signal processor via the matrix switch, after the set period of time; and float the return electrode of the neural interface not selected for neural sensing.

In some examples, the set period of time is greater than 10 microseconds.

In some examples, the controller is configured to select neural stimulation; select either the first or the second neural interface for neural stimulation; switchably couple the return electrode of the neural interface selected for neural stimulation to the current sink via the matrix switch; switchably couple all the first and second working electrodes to the voltage source via the matrix switch; and float the return electrode of the neural interface not selected for neural stimulation.

In some examples, the controller is configured to select neural sensing after neural stimulation; select either the first or the second neural interface for neural sensing; switchably couple the return electrode in the neural interface selected for neural sensing to the ground via the matrix switch; switchably couple all the first and second working electrodes to the ground via the matrix switch for a set period of time; switchably couple all the first and second working electrodes to an input of the signal processor via the matrix switch, after the set period of time; and float the return electrode of the neural interface not selected for neural sensing.

In some examples, the set period of time is greater than 10 microseconds.

In some examples, the controller is configured to select neural sensing of tissue; select either the first or the second neural interface for neural sensing; switchably couple the return electrode in the neural interface selected for neural sensing to the ground via the matrix switch; switchably couple all the first and second working electrodes to an input of the signal processor via the matrix switch; and float the return electrode of the neural interface not selected for neural sensing.

In some examples, the first neural interface includes at least one of a nerve cuff, a helical cuff, paddle electrodes, or an electrode array, and the second neural interface includes at least one of a nerve cuff, a helical cuff, paddle electrodes, or an electrode array.

According to an aspect, the technology relates to a method for configuring an implantable medical system, including providing a medical lead extending between a proximal end and first and second neural interfaces at respective first and second distal ends, the medical lead including a first and a second return conductor respectively extending from the proximal end of the medical lead to first and second return electrodes at first and second neural interfaces, and a working conductor respectively extending from the proximal end of the medical lead to first and second working electrodes in the first and second neural interfaces; providing a plurality of subsystems, including a current source, a current sink, a voltage source, a signal processor, and a ground, the signal processor having at least one input and at least one output; and providing a matrix switch configured to selectively couple any one of the first and second return electrodes to any one of the plurality of subsystems, and selectively couple all the first and second working electrodes to any one of the plurality of subsystems.

In some examples, the method includes selecting neural stimulation; selecting either the first or the second neural interface for neural stimulation; switchably coupling, via the matrix switch, the return electrode of the neural interface selected for neural stimulation to the ground; switchably coupling, via the matrix switch, the first and second working electrodes to the current source; and floating the return electrode of the neural interface not selected for neural stimulation.

In some examples, the method includes selecting neural sensing after neural stimulation; selecting either the first or the second neural interface for neural sensing; switchably coupling, via the matrix switch, all the electrodes to the ground for a set period of time greater than 10 microseconds; switchably coupling, via the matrix switch, the return electrode of the neural interface selected for neural sensing to the ground; switchably coupling, via the matrix switch, all the first and second working electrodes to the input of the signal processor; and floating the return electrode of the neural interface not selected for neural sensing.

In some examples, the method includes selecting neural sensing; selecting either the first or the second neural interface for neural sensing; switchably coupling, via the matrix switch, the return electrode of the neural interface selected for neural sensing to the ground; switchably coupling, via the matrix switch, all the first and second working electrodes to the input of the signal processor; and floating the return electrode of the neural interface not selected for neural sensing.

In some examples, the first neural interface includes at least one of a nerve cuff, a helical cuff, paddle electrodes, or an electrode array, and the second neural interface includes at least one of a nerve cuff, a helical cuff, paddle electrodes, or an electrode array.

This Summary section introduces some features of nonlimiting and non-exhaustive examples of the present disclosure, and is not intended to limit the scope of the claims.

### BRIEF DESCRIPTION OF THE DRAWINGS

The drawings, together with the specification, illustrate nonlimiting and non-exhaustive example embodiments of the present disclosure.

FIG. 1 is a block diagram of a medical system according to one or more embodiments.

FIG. 2 is a block diagram of a stimulation system according to one or more embodiments.

FIG. 3 is a block diagram of a system including a medical lead with selective connections to various subsystems according to one or more embodiments.

FIG. 4 depicts a circuit diagram of electrical connections of electrodes of the first and second cuff electrodes of the stimulation device of FIG. 1 according to one or more embodiments.

FIG. 5 is a flow chart for a method for coupling of a medical lead to a selected neural interface according to one or more embodiments.

FIG. 6 depicts the stimulation system of FIG. 1 when implanted in a body.

FIG. 7 depicts another stimulation system according to one or more embodiments and when implanted in a body.

FIG. 8 is a block diagram of a portion of an implantable medical system including a medical lead with selective connections to subsystems according to one or more embodiments.

FIG. 9 is a flowchart for a method for configuring a medical lead for either neural stimulation or sensing according to one or more embodiments.

FIG. 10 is a flowchart for a method for configuring a selected neural interface for neural stimulation according to one or more embodiments.

FIG. 11 is a flowchart for a method for configuring a selected neural interface for neural sensing according to one or more embodiments.

### DETAILED DESCRIPTION

Nonlimiting and non-exhaustive embodiments of stimulation devices and of methods of stimulation will now be described in more detail with reference to the drawings.

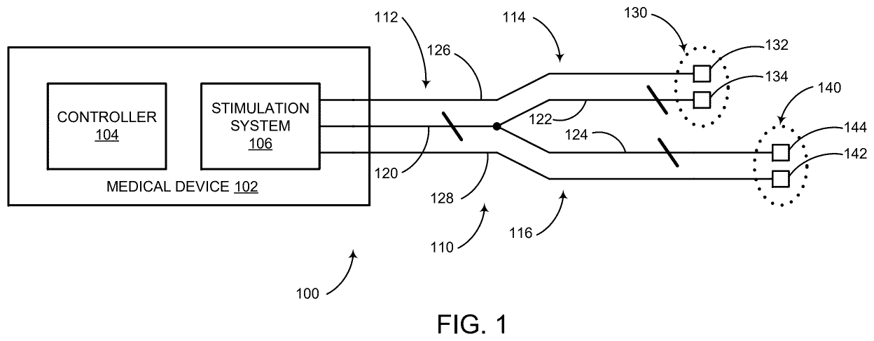

FIG. 1 is a block diagram of medical system 100 for generating neural stimulation signals applied to one or two nerves via neural interfaces 130 or 140, according to one or more embodiments. Neural interfaces 130 and 140 may be nerve cuffs, paddle electrodes, electrode arrays or other interfaces used for stimulating nerves. Medical system 100 includes medical device 102 connected to implantable medical lead 110. Medical device 102 may be an implantable pulse generator (IPG) and includes controller 104 and stimulation system 106. Controller 104 may include nonvolatile memory storing tissue stimulation protocols determined by a clinician using a clinician programmer for a patient. The tissue stimulation protocols may also be selected by a patient using a patient remote from a preselected set determined by a clinician. Controller 104 generates control signals for stimulation system 106 based on a selected stimulation protocol and selects either neural interface 130 or 140 to receive stimulation signals at any given time. Not shown in FIG. 1 are other systems typically part of implantable medical devices, such as systems to receive wireless power transmissions from external power transmitters, power supplies and systems to communicate with patient remotes and clinician programmers. However, it will be understood that some embodiments of medical systems described herein may include such other systems.

Medical lead 110 has a main section 112, which then splits into two branches 114 and 116. Branch 114 connects to neural interface 130 at one distal end and branch 116 connects to neural interface 140 at another distal end. Main section 112 has N+2 conductors at its proximal end and is connected to stimulation system 106 in medical device 102, where N can be any integer greater than or equal to one. Branch 114 has conductor 126 connected to reference electrode 132 in neural interface 130 and also N conductors 120 connected to N working electrodes 134 in neural interface 130. Branch 116 has conductor 128 connected to reference electrode 142 in neural interface 140 and also N conductors 120 connected to N working electrodes 144 in neural interface 140.

Stimulation system 100 may be a subcutaneously implantable stimulation device and include an implantable pulse generator (IPG) and one or more leads (e.g., electrical leads) electrically coupled to the IPG and configured to provide stimulation (e.g., electrical stimulation). The stimulation device 100 may be implanted in a body (e.g., a human or a non-human animal) and utilized to stimulate, for example, one or more nerves to treat one or more conditions. For example, the stimulation system 100 may be utilized to stimulate two hypoglossal nerves on opposite sides of a sagittal plane of the body in order to treat obstructive sleep apnea (OSA), including OSA where complete concentric collapse (CCC) occurs. In another example, the stimulation system 100 may be used to selectively stimulate another nerve (e.g., a vagus nerve and/or other nerves) in addition to, or instead of, the hypoglossal nerve to treat one or more medical conditions.

The one or more leads may include, for example, a bifurcated lead including a common lead 110 electrically coupled to the medical device 102 at a proximal end, a first branch 114 branching off from a distal end of the common lead 110, and a second branch 116 branching off from the distal end of the common lead 110. The first branch 114 may include a first neural interface 130, such as a cuff electrode, and the second branch 116 may include a second neural interface 140, such as a cuff electrode.

However, the present disclosure is not limited thereto. For example, the lead may be a multi-furcated lead that includes two or more branches or sub-leads that, for example, branch off from a common lead and each include one or more electrodes. The one or more electrodes on each sub-lead may be configured (shaped, sized, relatively positioned, relatively oriented, and/or of a number) to stimulate one or more nerves (e.g., the proximal and/or distal portion of the hypoglossal nerve). Because the shape, size, and position of nerves vary, the shape, size, relative positions, relative orientations, and/or number of the one or more electrodes along each branch or sub-lead may vary based on the particular nerve that the one or more electrodes are configured to stimulate. Moreover, in one or more embodiments, each branch or sub-lead may include one or more stimulators other than electrodes, such as one or more coils for generating a time-varying magnetic field, one or more acoustic stimulators, etc.

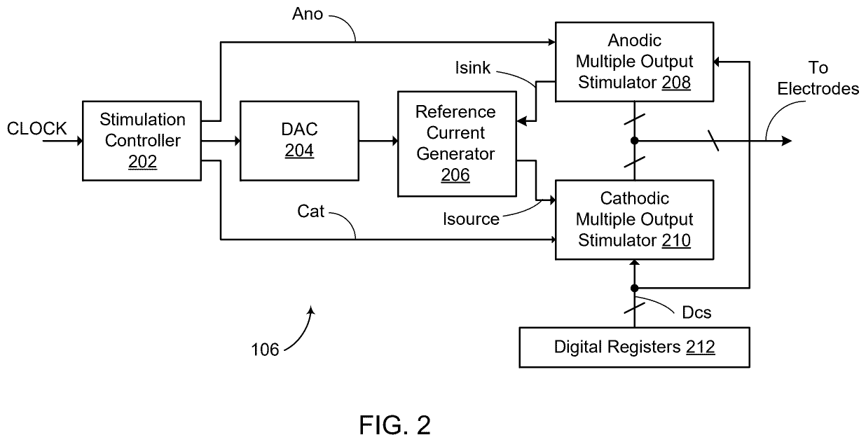

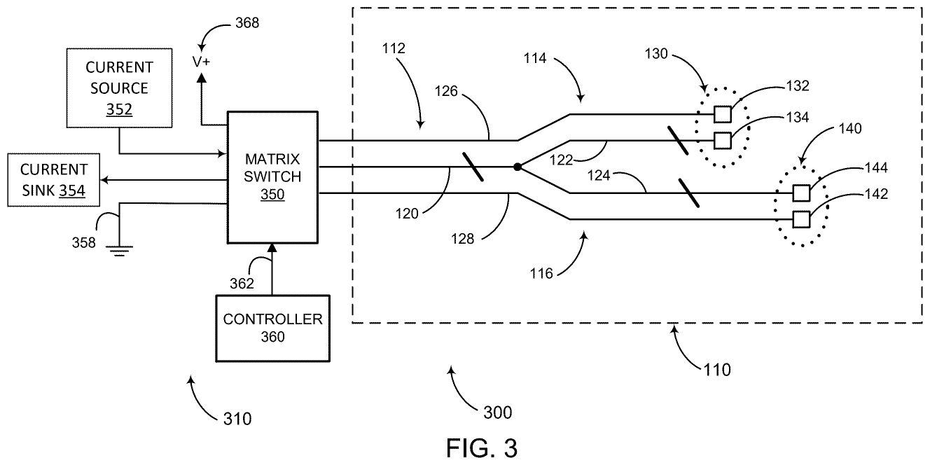

FIG. 2 is a block diagram of an exemplary stimulation system 106 connected by switches to the conductors of medical lead 110 according to one or more embodiments. Stimulation system 106 is similar to the pulse generator shown in FIG. 3 in U.S. Pat. No. 9,446,241. Stimulation controller 202 receives control signals from controller 104 via signal lines, which are not shown in FIG. 2. Digital control signals from stimulation controller 202 are sent to anodic stimulator 208, cathodic stimulator 210 and digital to analog converter DAC 204. Stimulation system 106 provides for selecting either neural interface 130 or 140 and for selecting an anodic or cathodic stimulator based upon tissue stimulation requirements determined by a clinician.

The outputs of the anodic stimulators 208 and cathodic stimulators 210 are selected by stimulation controller 202 by setting the corresponding “bits” in digital registers 212. Control lines from stimulation controller 202 to digital registers 212 are not shown in FIG. 2. Digital registers 212 generate digital control signals Dcs, which control the selection of either neural interface 130 or 140 to provide stimulation signals to tissue by selecting via switches either of the respective reference electrodes 132 or 142 via the selection of either respective conductor 126 or 128 of medical lead 110.

Digital registers 212 also store information regarding stimulation pulse duration, amplitude and profile as well as other operational parameters. Based upon information stored in digital registers 212 and the Clock signal, stimulation controller 202 generates the desired stimulation pulse amplitude and triggers digital to analog converter DAC 204 to generate an output. Based upon the DAC 204 output, reference current source generator 206 provides a current sink for Isink current for the anodic stimulator 208 and provides a current source Isource current for the cathodic stimulator 210. Stimulation controller 202 generates control signal Ano to turn on the anodic stimulator 208 to output anodic current at one or more selected outputs according to the programmed anodic pulse amplitude, duration and profile. Anodic stimulator 208 may include one or more normally open switches. Similarly, stimulation controller 202 also generates control signal Cat to turn on cathodic stimulator 210 to output cathodic current at one or more selected outputs according to the programmed cathodic pulse amplitude, duration and profile. Stimulation system 106 is connected to medical lead 110.

FIG. 3 is a block diagram of a system 300 including medical lead 110 with switchable selective connections via matrix switch 350 to various subsystems 310 including current source 352, current sink 354, voltage source 368 and ground 358 according to one or more embodiments. System 300 includes medical lead 110 connected to subsystems 310, which is a portion of a stimulation system with control signals coming from controller 360. Controller 360 generates control signals for the stimulation system based on a selected stimulation protocol and selects either neural interface 130 or 140 to receive stimulation signals.

In one or more embodiments, stimulation controller 360 may select neural interface 130 or 140 for tissue stimulation and can generate control signals on control line 362 to matrix switch 350 to connect current source 352 to the N conductors 120 to the working electrodes 134 and 144. Controller 360 can also generate control signals on control line 362 to matrix switch 350 to connect ground 358 to reference electrode 132 via conductor 126 to enable neural interface 130 for stimulation of neural tissue.

In other embodiments, controller 360 can generate control signals on control line 362 to matrix switch 350 to connect ground 358 to reference electrode 142 via conductor 128 to enable neural interface 140 for stimulation of neural tissue Conductor 120 may have N conductors, where N is an integer greater than or equal to one. As a result of switching functions provided by matrix switch 350 either neural interface 130 or 140 may be activated to stimulate tissue.

When neural interface 130 is delivering stimulation signals to tissue, neural interface 140 is not functional, because reference electrode 142 is floating, since it is not connected to ground or a power source. Neural interfaces 130 and 140 are at separate distal ends of medical lead 110 and separated by a distance sufficient to prevent reference electrode 142 from acting as a reference electrode to working electrodes 134 because of a relatively high impedance path between reference electrode 142 and working electrodes 134.

In one or more embodiments, stimulation controller may select neural interface 130 or 140 for tissue stimulation and can generate control signals via control line 362 to matrix switch 350 to connect voltage source 368 to working electrodes 134 and 144 via N conductors 120. Controller 360 can also generate control signals via control line 362 to matrix switch 350 to connect current sink 354 to reference electrode 142 via conductor 128 to activate neural interface 140 for stimulation of neural tissue.

In other embodiments, controller 360 can generate control signals via control line 362 to matrix switch 350 to connect current sink 354 to reference electrode 132 via conductor 126 to activate neural interface 130 for stimulation of neural tissue. Conductor 120 may have N conductors, where N is an integer greater than or equal to one.

When neural interface 140 is delivering stimulation signals to tissue, neural interface 130 is not functional, because reference electrode 132 is floating, since it is not connected to ground or a power source. Neural interfaces 130 and 140 are at separate distal ends of medical lead 110 and separated by a distance sufficient to prevent reference electrode 132 from acting as a reference electrode to working electrodes 144 because of a relatively high impedance path between reference electrode 132 and working electrodes 144.

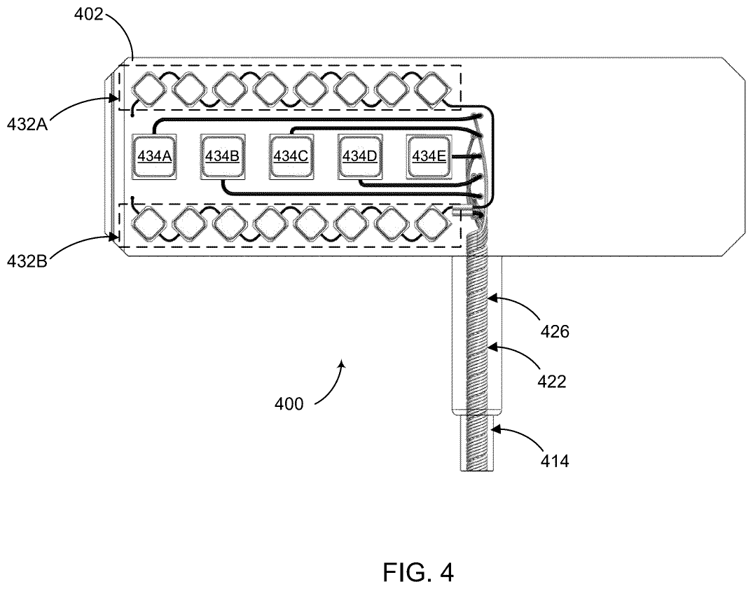

FIG. 4 depicts a nerve cuff 400 connected to branch 414 of a medical lead according to one or more embodiments. Branch 414 includes N conductors 422 to working electrodes 434A-434E and conductor 426 to arrays of reference electrodes 432A and 432B. The electrodes are disposed on flexible base 402. N can be an integer equal to or greater than one. In some embodiments, working electrodes 434A-434E can be connected together as one electrode. In some embodiments, working electrodes 434A-434E can each be connected to separate conductors within the N conductors 422 of branch 414, and provide separate N channels of stimulation signals.

Medical system 100, in one or more embodiments, includes medical device 102 and medical lead 110 and can be configured to be connected to one nerve cuff, like nerve cuff 400, at a first distal end and functioning as neural interface 130 and connected to a second nerve cuff, like nerve cuff 400, at a second distal end and functioning as neural interface 140.

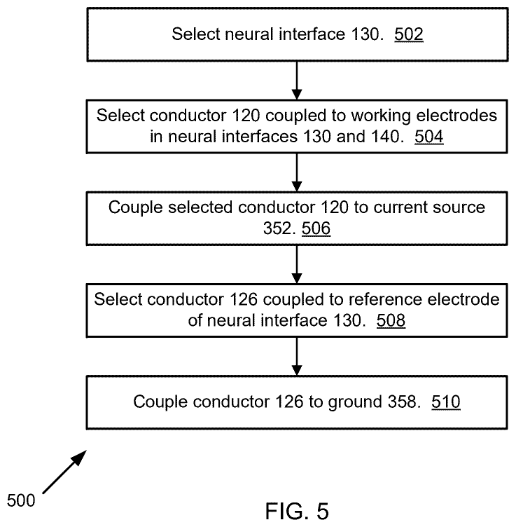

FIG. 5 is a flow chart for method 500 for selective coupling of medical lead 110 to neural interface 130 for system 300 in FIG. 3 according to one or more embodiments. Step 502 of the method selects neural interface 130 to receive stimulation signals. Step 504 of the method selects working electrodes 134 and 144 via conductor 120. Step 506 connects conductor 120 to current source 352. Step 508 selects conductor 126 coupled to reference electrode 132 of neural interface 130. Step 510 connects conductor 126 and reference electrode 132 to ground 358. Neural interface 130 is coupled to current source 352 and ground 358 and is configured to receive stimulation signals from medical device 102.

At step 510, neural interface 140 is not functional because reference electrode 142 of neural interface 140 is floating since it is not coupled to ground or a power source. Reference electrode 142 cannot function as a reference electrode to working electrodes 134 because of a relatively high impedance path between reference electrode 142 and working electrodes 134.

Method 500 provides steps for the stimulation during a first time period of tissue using neural interface 130, while neural interface 140 is not functional.

When both work and return electrodes on a same lead are driven together, stimulation provided by the lead to a nerve in proximity to the lead may be, for example, bipolar or tripolar stimulation, because both anode and cathode electrodes on the same lead are driven concurrently (e.g., simultaneously). In contrast, when only one of work or return electrodes on a same lead are driven (without the other), then the stimulation provided by the lead may be monopolar stimulation, because only one of cathode electrodes or anode electrodes are driven (without the other). Monopolar stimulation is generally much more diffuse and can be insufficient in intensity to stimulate the nearby nerve.

The method 500 for providing stimulation using the neural interface 130 without providing stimulation using the neural interface 140 may define a first stimulation mode of the stimulation system 106. A corresponding method may be used by the stimulation system 106 to provide stimulation using the neural interface 140 without providing stimulation using the neural interface 130, and this corresponding method may define a second stimulation mode of the stimulation system 106. In some embodiments, the stimulation system 106 is configured to utilize the second stimulation mode during a second time period after (e.g., immediately after) the first time period.

The controller 104 may be configured, when executing instructions stored in the memory, not shown in the figures, to control the stimulation system 106 to alternatingly (e.g., alternatingly at a set frequency, such as a frequency equal to or greater than 1 Hz, 10 Hz, 60 Hz, or 100 Hz) utilize the first and second stimulation modes to drive the first and second branches 114 and 116. For example, the controller 104 may be configured to control the stimulation system 106 during a third time period after (e.g., immediately after) the second time period to drive the first and second branches 114 and 116 according to the first stimulation mode.

The present disclosure is not limited by the stimulation device 100 and stimulation methods of FIGS. 1-5. Although the stimulation device 100 is illustrated to include two branches, the present disclosure is not limited thereto. The stimulation device 100 may include, for example, three or more branches (e.g., each branching off from the common lead 110), and each branch may include, for example, one or more working electrodes (e.g., 1, 2, 3, 4, 5, or more working electrodes) and one or more return electrodes (e.g., 1, 2, 3, 4, 5, or more return electrodes).

In one or more embodiments, the controller 104 may be configured, when executing instructions stored in memory, to control the stimulation system 106 to sequentially drive return electrodes on the sub leads so that only one of the three or more branches has its respective return electrode(s) driven at a time, while the return electrode(s) of the remaining sub leads are not driven, in a similar manner as is described herein above. For example, the stimulation system 100 may include three branches, and the stimulation system 106 may drive three branches in a first stimulation mode during a first time period, drive the three branches in a second stimulation mode during a second time period after the first time period, drive the three branches in a third stimulation mode during a third time period after the second time period, and drive the three branches in the first stimulation mode during a fourth time period after the third time period.

In one or more embodiments, a single bifurcated lead (or a single branch) may include the first and second neural interfaces 130 and 140 respectively at two positions along the lead that are separated from each other.

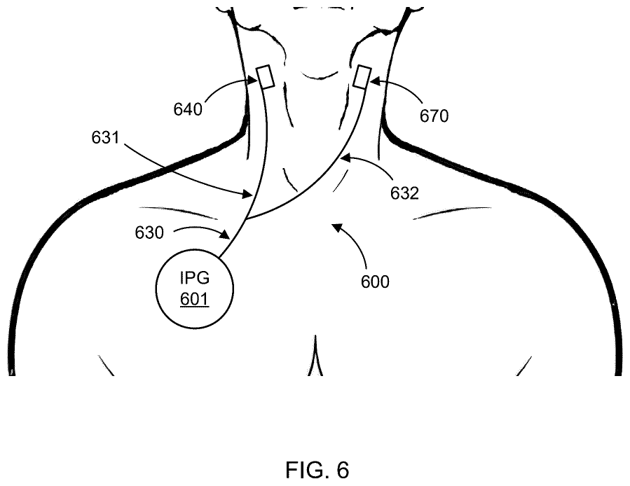

Referring to FIG. 6, the stimulation device 600 may be an implantable stimulation device, for example, for providing stimulation to multiple nerves and/or muscles to treat one or more medical conditions. The stimulation device 600 (e.g., the housing 601 and at least the exterior surfaces of the common lead 630, the first branch or sub lead 631, and the second branch or sub lead 632) may include (e.g., may be made of) a biocompatible material. The work and return electrodes on each sub lead may each be positioned within the body proximal to (e.g., be around or at least partially surrounding) a target nerve and/or muscle, such as a proximal or distal portion of the HGN or other nerve. For example, the first and second cuff electrodes 640 and 670 may be respectively positioned to stimulate first and second HGN nerves on opposite sides of a sagittal plane of the body.

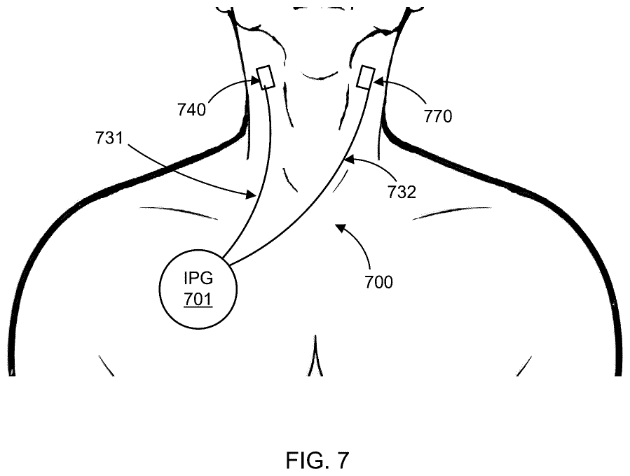

FIG. 7 depicts another stimulation device 700 according to one or more embodiments and when implanted in a body. The stimulation device 700 may include features similar to, or the same as, the stimulation device 600 shown in FIG. 6. For example, the stimulation device 700 may include an IPG including a housing 701 and a power source, a power modulation electronics, a driver, a memory, and a controller within the housing 701. The stimulation device 700 may include a first lead 731 including a first cuff electrode 740, and a second lead 732 including a second cuff electrode 770. The stimulation device 700 may differ from the first stimulation device 600 in that the first and second leads 731 and 732 are separately coupled to the IPG, instead of branching off from a common lead that is coupled to the IPG.

In one or more embodiments a stimulation device and/or a method of stimulation may be utilized to treat one or more medical conditions by stimulating two or more nerves. For example, the OSA (including OSA where complete concentric collapse occurs), dysphagia, etc. may be treated. OSA where complete concentric collapse occurs may be treated, for example, by stimulating (e.g., alternatingly stimulating) two HGN nerves on opposite sides of a sagittal plane. As another example, the stimulation device and/or a method of stimulation may be utilized to treat epilepsy or depression, for example, by stimulating the vagus nerve at two or more discrete locations or in conjunction with another nerve.

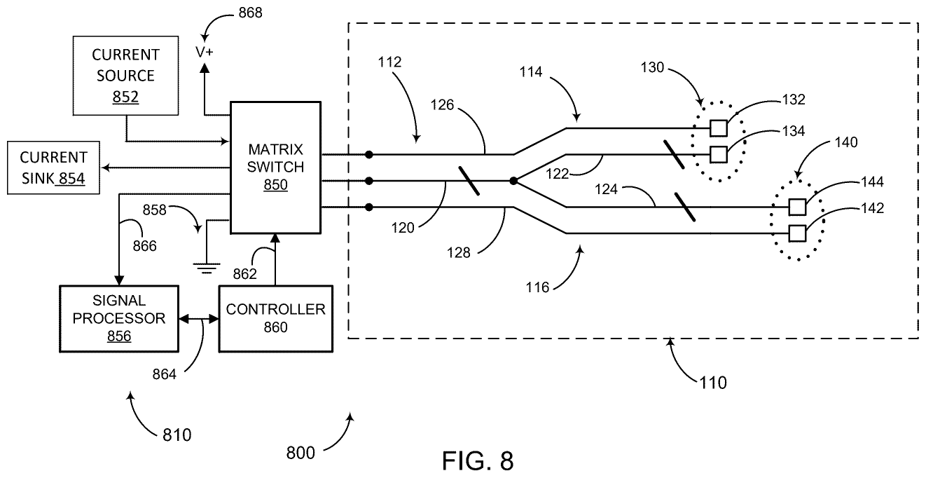

FIG. 8 is a block diagram of a portion of an implantable medical system 800 including medical lead 110 with connections to various subsystems 810 via matrix switch 850 according to one or more embodiments. Medical lead 110 shown in FIG. 8 is similar to medical lead 110 as described previously with respect to FIG. 1. One side of matrix switch 850 connects to the conductors and their associated electrodes at the proximal end 112 of medical lead 110. The other side of matrix switch 850 connects to various subsystems 810 including: voltage source 868, current source 852, current sink 854, signal processor 856, and ground 858. The signal processor 856 is coupled to the matrix switch 850 via input 866. The operation of matrix switch 850 is performed by controller 860 via control lines 862. Bidirectional link 864 provides control lines to signal processor 856 from controller 860 and data lines from signal processor 856 to controller 860.

Depending on the use of the medical system, at any given time, at least two of the various subsystems may be connected to the working conductor 120 and one of the reference conductors 126 or 128. In some examples, the medical system is to be used for neural stimulation and the working conductor 120 is connected to current source 852 and either the reference conductor 126 or 128 is connected to the ground 858. In some examples, the medical system is to be used for neural stimulation and the working conductor 120 is connected to voltage source 868 and either the reference conductor 126 or 128 is connected to the current sink 854.

In some examples, the medical system is to be used for neural sensing and the working conductor 120 is connected to the signal processor 856 and either the reference conductor 126 or 128 is connected to the ground 858.

Matrix switch 850 can also not connect any of the three available conductors and their associated electrodes in medical lead 110, so that the unconnected conductor is considered to be electrically floating, since that conductor and its associated electrode are not connected to a ground, a power source or any other electronic system.

Controller 860 has bidirectional links 864 with signal processor 856 to regulate one or more operating parameters of signal processor 856, such as, for example, the analog to digital conversion of the sensed neurological signals, such as sampling rate, analog and/or digital filtering, analysis by a neural network, analysis by a machine learning process and other parameters. The processed digital neural data from signal processor 856 can also be sent to controller 860 via link 864 for further processing by controller 860, and controller 860 may use such data to determine future neural stimulation protocols and neural sensing protocols. For example, the controller 860 may determine or set one or more stimulation parameters and/or a stimulation protocol based at least in part on the processed digital neural data. Signal processor 856 and controller 860 may each have non-volatile storage systems for the recording of sensed neural data.

System 800 provides several modes of operation of the neural interfaces 130 and 140, including:
A. neural stimulation of tissue using neural interface 130,
B. neural stimulation of tissue using neural interface 140,
C. neural sensing of tissue using neural interface 130, or
D. neural sensing of tissue using neural interface 140.

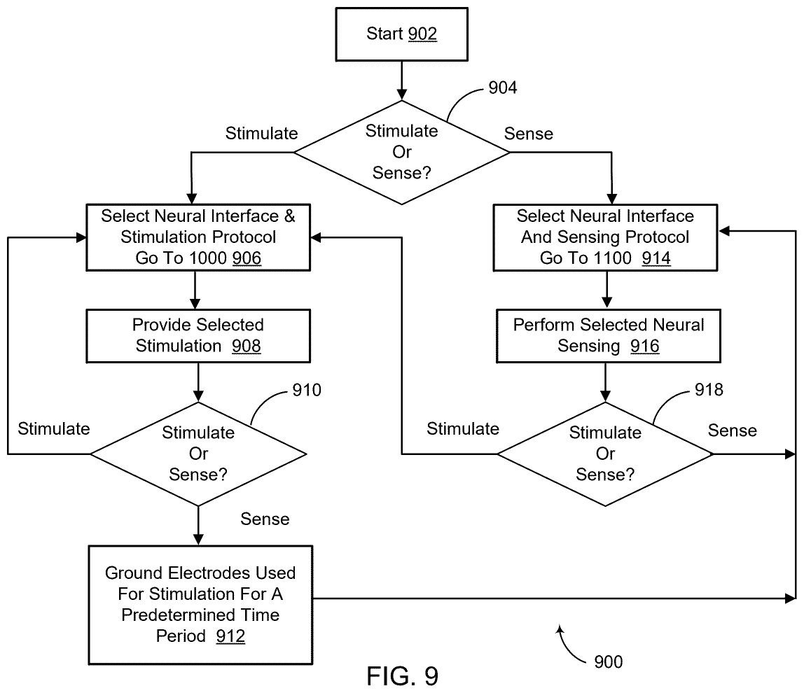

FIG. 9 is a flowchart of method 900 for configuring medical system 800 of FIG. 8 including medical lead 110 for either neural stimulation or sensing according to one or more embodiments under the management of controller 860. Method 900 starts at 902 and proceeds to decision step 904. Step 904 includes a decision as to whether the method proceeds to stimulate a nerve or sense neurological activity. If the decision is to stimulate, the method proceeds to block 906. If the decision is to sense neural activity, then the method proceeds to block 914.

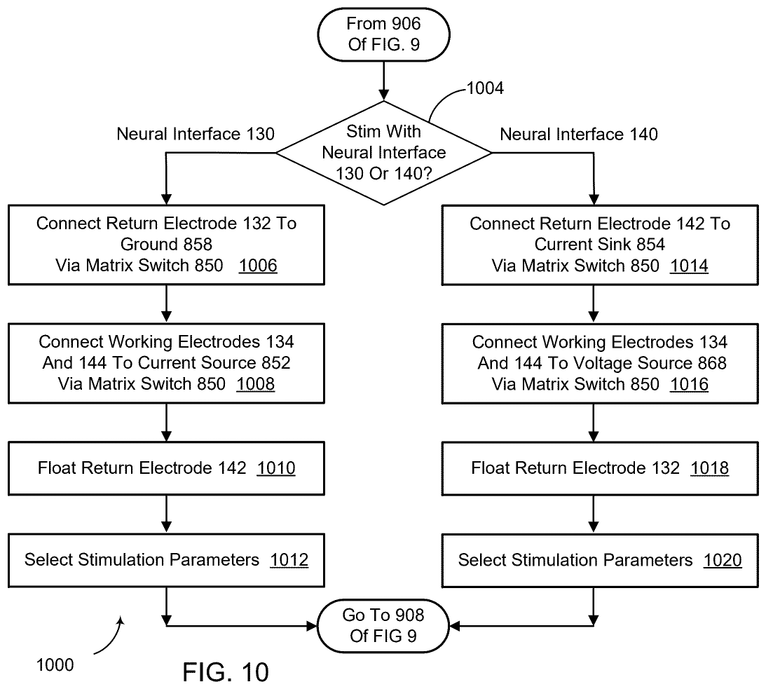

Step 906 of method 900 includes the selection of either neural interface 130 or 140 and a stimulation protocol, which are described in more detail with respect to method 1000 shown in and described herein with respect to FIG. 10. After step 906, the method proceeds to step 908, to provide the selected neural interface with the selected neural stimulation. After delivering stimulation in step 908, method 900 proceeds to decision step 910, where the method chooses to perform either neural stimulation or neural sensing.

If the decision in step 910 of method 900 is to provide more stimulation, then method 900 proceeds to step 906. If the decision in step 910 is to provide neural sensing, the method 900 proceeds to step 912, and all the electrodes used for stimulation are grounded for a predetermined period of time, such as time period equal to or greater than 10 microseconds.

In step 912, the grounding of the return electrodes 132 or 142 and working electrodes 134 and 144 is provided by the connection of the proximal ends of the respective conductors, such as return conductors 126 or 128 and working conductor 130 to the ground 858 via matrix switch 850 as managed by controller 860. The grounding of the stimulating electrodes after stimulation has been delivered, provides for discharging any residual electrical charges that may still be on the electrodes due to neural stimulation. Grounding the stimulating electrodes prepares them for sensing neural signals.

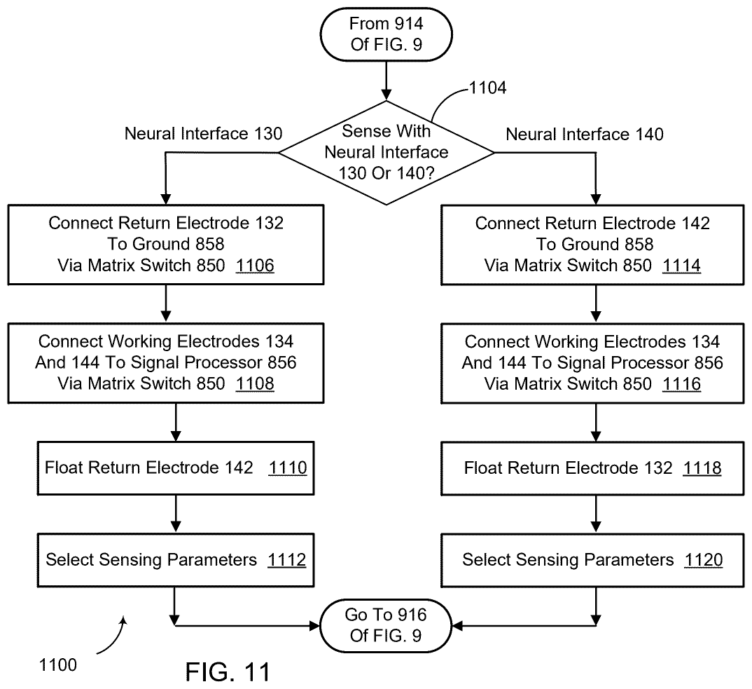

After the electrodes have been grounded, the method 900 proceeds to step 914, and the method will select either neural interface 130 or 140 for neural sensing and a sensing protocol is selected. The steps to perform 914, according to some examples, are shown and explained as method 1100 with respect to FIG. 11.

After step 914, method 900 proceeds to step 916 and the method proceeds to provide neural sensing as determined in step 914.

After step 916, method 900 proceeds to decision step 918 and the method chooses between neural stimulation or sensing. If the decision in step 918 of method 900 is to provide more stimulation, then method 900 proceeds to step 906. If the decision in step 918 is to provide neural sensing, the method 900 proceeds to step 914.

FIG. 10 is a flowchart for method 1000 for selecting and configuring either neural interface 130 or 140 for neural stimulation according to one or more embodiments. Method 1000 represents the steps performed in step 906 of FIG. 9. Step 1004 of the method is a decision, whether to stimulate with neural interface 130 or 140. If neural interface 130 is selected, then the method proceeds to step 1006. If neural interface 140 is selected, then the method proceeds to step 1014.

In step 1006, method 1000 connects return electrode 132 and return conductor 126 to ground 858 via matrix switch 850 and the method proceeds to step 1008. In step 1008, method 1000 connects working electrodes 134 and 144 and working conductor 120 to current source 852 via matrix switch 850 and the method proceeds to step 1010. In step 1010, method 1000 floats return electrode 142 and return conductor 128 and the method proceeds to step 1012.

In other embodiments of steps 1006 and 1008, matrix switch 850 can connect return electrode 132 and return conductor 126 to current sink 854 and working electrodes 134 and 144 and working conductor 120 to voltage source 868.

In step 1012, method 1000 selects one or more stimulation parameters such as at least one of current, voltage, polarity, pulse width, pulse waveform, pulse train length, frequency, or time duration, and the method proceeds to step 908, which has been described with respect to FIG. 9.

In step 1014, method 1000 connects return electrode 142 and return conductor 128 to current sink 854 via matrix switch 850 and the method proceeds to step 1016. In step 1016, method 1000 connects working electrodes 134 and 144 and working conductor 120 to voltage source 868 via matrix switch 850 and the method proceeds to step 1018.

In some other embodiments of steps 1014 and 1016, matrix switch 850 can connect return electrode 142 and return conductor 128 to ground 858 and working electrodes 134 and 144 and conductor 120 to current source 852.

In step 1018, method 1000 floats return electrode 132 and return conductor 126 and the method proceeds to step 1020.

In step 1020, method 1000 selects one or more stimulation parameters such as at least one of current, voltage, polarity, pulse width, pulse waveform, pulse train length, frequency, or time duration, and the method proceeds to step 908, which has been described with respect to FIG. 9.

FIG. 11 is a flowchart for a method 1100 for selecting and configuring either neural interface 130 or 140 for neural sensing according to one or more embodiments. Method 1100 represents the steps performed in step 914 of FIG. 9.

Step 1104 of method 1100 selects either neural interface 130 or 140 for neural sensing. If neural interface 130 is selected, the method proceeds to step 1106. If neural interface 140 is selected, the method proceeds to step 1114.

Step 1106 of method 1100 connects return electrode 132 and return conductor 126 to ground 858 via matrix switch 850, and the method proceeds to step 1108. Step 1108 of the method connects working electrodes 134 and 144 and conductors 120 to the input 866 of signal processor 856 via matrix switch 850 and the method proceeds to step 1110. Step 1110 floats return electrode 142 and return conductor 128, and the method proceeds to step 1112. Although electrodes 132 and 142 and conductors 126 and 128 are referred to herein as “return” elements in the context of neural stimulation, these “return” elements can be understood as “reference” elements in the context of neural sensing.

Step 1112 of method 1100 selects one or more sensing parameters, such as the time duration of neural sensing and/or the processing of the sensed neural signals by the connected signal processor 856. After the one or more sensing parameters are selected, the method proceeds to step 916 of method 900 in FIG. 9.

Step 1114 of method 1100 connects return electrode 142 and return conductor 128 to ground 858 via matrix switch 850, and the method proceeds to step 1116. Step 1116 of the method connects working electrodes 134 and 144 and conductor 120 to the input 866 of signal processor 856 via matrix switch 850, and the method proceeds to step 1118. Step 1118 of the method floats return electrode 132 and return conductor 126 and the method proceeds to step 1120.

Step 1120 of method 1100 selects one or more sensing parameters, such as the time duration of neural sensing and/or the processing of the sensed neural signals by the connected signal processor 856. After the one or more sensing parameters are selected, the method proceeds to step 916 of method 900 in FIG. 9.

Although some methods for performing stimulation and sensing have been discussed with reference to FIGS. 5 and 9-11, the present disclosure is not limited thereto. Devices for providing stimulation and sensing, and processes performed by such devices, have been described herein with reference to FIGS. 1-11, and the present disclosure includes all methods for providing stimulation and/or sensing that include any combination of such processes in any suitable order.

It will be understood that, although the terms “first”, “second”, “third”, etc., may be utilized herein to describe one or more suitable features, elements, or processes, these features, elements, or processes should not be limited by these terms. These terms are only utilized to distinguish one feature, element, or process from another feature, element, or process. Thus, a first feature, element, or process discussed herein could be termed a second feature, element, or process without departing from the spirit and scope of the present disclosure.

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. It will be further understood that the terms “comprises,” “comprising,” “includes,” and “including,” when utilized in this specification, specify the presence of stated features, elements, and/or processes, but do not preclude the presence or addition of one or more other features, elements, and/or processes. As utilized herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element or feature is referred to as being “on” or “coupled to” another element or feature, it can be directly on or coupled to the other element or feature, or one or more intervening element(s) or feature(s) may be present. In contrast, when an element or feature is referred to as being “directly on,” or “directly coupled to” another element or feature, there are no intervening elements or features present.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The stimulation devices (e.g., the stimulation system 100) and/or any relevant components of the stimulation devices (e.g., the controller 104, the memory, the stimulation system 106, etc.) within the scope of the present disclosure may be implemented utilizing any suitable circuits, hardware (e.g. discrete electronic components or an application-specific integrated circuit), firmware, software, or a combination of software, firmware, hardware, and circuits. For example, the one or more suitable components of the stimulation devices may be formed on one integrated circuit (IC) chip or on separate IC chips. Further, the one or more suitable components of the stimulation devices may be implemented on a flexible printed circuit film, a tape carrier package (TCP), a printed circuit board (PCB), or formed on one substrate. Further, the one or more suitable components of the stimulation devices may be a process or thread, running on one or more processors, in one or more computing devices, executing computer program instructions and interacting with other system components for performing the one or more suitable functionalities described herein. The computer program instructions may be stored in a memory which may be implemented in a computing device utilizing a standard memory device, such as, for example, a random access memory (RAM). The computer program instructions may also be stored in other non-transitory computer readable media such as, for example, a CD-ROM, flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although specific embodiments are described herein, the scope of the technology is not limited to those specific embodiments. Moreover, while different examples and embodiments may be described separately, such embodiments and examples may be combined with one another in implementing the technology described herein. One skilled in the art will recognize other embodiments or improvements that are within the scope and spirit of the present technology. Therefore, the specific embodiments illustrated and described herein are only examples, and the scope of the present disclosure encompasses additional embodiments. The scope of the technology is defined by the following claims and any equivalents thereof.

What is claimed is:

1. An implantable medical system comprising:
a medical lead comprising:
a first return conductor extending from a proximal end of the medical lead to a first return electrode at a first neural interface at a first distal end of the medical lead,
a second return conductor extending from the proximal end of the medical lead to a second return electrode at a second neural interface at a second distal end of the medical lead, and
a working conductor extending from the proximal end of the medical lead to first and second working electrodes respectively in the first and second neural interfaces; and
a plurality of subsystems comprising a current source, a current sink, a signal processor, a voltage source, and a ground,
wherein the signal processor has at least one input and at least one output.

2. The implantable medical system of claim 1, wherein:
the first neural interface is configured for either neural stimulation or neural sensing; and
the second neural interface configured for either neural stimulation or neural sensing.

3. The implantable medical system of claim 1, further comprising a matrix switch configured to:
selectively couple any one of the first and second return electrodes to any one of the plurality of subsystems; and
selectively couple all the first and second working electrodes to any other one of the plurality of subsystems.

4. The implantable medical system of claim 3, wherein any one of the electrodes can float by not being coupled via the matrix switch to any one of the plurality of subsystems.

5. The implantable medical system of claim 3, further comprising a controller configured to control the operation of the matrix switch.

6. The implantable medical system of claim 5, wherein the controller is configured to select one of the first neural interface and the second neural interface for either neural stimulation or neural sensing.

7. The implantable medical system of claim 6, wherein the controller is configured to select the return electrode of the other one of the first neural interface and the second neural interface to be floating.

8. The implantable medical system of claim 5, wherein the controller is configured to:
select neural stimulation;
select either the first or the second neural interface for neural stimulation;
switchably couple the return electrode of the neural interface selected for neural stimulation to the ground via the matrix switch;
switchably couple all the first and second working electrodes to the current source via the matrix switch; and
float the return electrode of the neural interface not selected for neural stimulation.

9. The implantable medical system of claim 8, wherein the controller is configured to:
select neural sensing after neural stimulation;
select either the first or the second neural interface for neural sensing;
switchably couple the return electrode of the neural interface selected for neural sensing to the ground via the matrix switch;
switchably couple all the first and second working electrodes to the ground via the matrix switch for a set period of time;
switchably couple all the first and second working electrodes to an input of the signal processor via the matrix switch, after the set period of time; and
float the return electrode of the neural interface not selected for neural sensing.

10. The implantable medical system of claim 9, wherein the set period of time is greater than 10 microseconds.

11. The implantable medical system of claim 5, wherein the controller is configured to:
select neural stimulation;
select either the first or the second neural interface for neural stimulation;
switchably couple the return electrode of the neural interface selected for neural stimulation to the current sink via the matrix switch;
switchably couple all the first and second working electrodes to the voltage source via the matrix switch; and
float the return electrode of the neural interface not selected for neural stimulation.

12. The implantable medical system of claim 11, wherein the controller is configured to:
select neural sensing after neural stimulation;
select either the first or the second neural interface for neural sensing;
switchably couple the return electrode in the neural interface selected for neural sensing to the ground via the matrix switch;
switchably couple all the first and second working electrodes to the ground via the matrix switch for a set period of time;
switchably couple all the first and second working electrodes to an input of the signal processor via the matrix switch, after the set period of time; and
float the return electrode of the neural interface not selected for neural sensing.

13. The implantable medical system of claim 12, wherein the set period of time is greater than 10 microseconds.

14. The implantable medical system of claim 5, wherein the controller is configured to:
select neural sensing of tissue;
select either the first or the second neural interface for neural sensing;
switchably couple the return electrode in the neural interface selected for neural sensing to the ground via the matrix switch;
switchably couple all the first and second working electrodes to an input of the signal processor via the matrix switch; and
float the return electrode of the neural interface not selected for neural sensing.

15. The implantable medical system of claim 1, wherein the first neural interface comprises at least one of a nerve cuff, a helical cuff, paddle electrodes, or an electrode array, and the second neural interface comprises at least one of a nerve cuff, a helical cuff, paddle electrodes, or an electrode array.

16. A method for configuring an implantable medical system, comprising:
providing a medical lead extending between a proximal end and first and second neural interfaces at respective first and second distal ends, the medical lead comprising:
a first and a second return conductor respectively extending from the proximal end of the medical lead to first and second return electrodes at first and second neural interfaces, and
a working conductor respectively extending from the proximal end of the medical lead to first and second working electrodes in the first and second neural interfaces;
providing a plurality of subsystems, comprising a current source, a current sink, a voltage source, a signal processor, and a ground, the signal processor having at least one input and at least one output; and
providing a matrix switch configured to:
selectively couple any one of the first and second return electrodes to any one of the plurality of subsystems, and
selectively couple all the first and second working electrodes to any other one of the plurality of subsystems.

17. The method of claim 16, further comprising:
selecting neural stimulation;
selecting either the first or the second neural interface for neural stimulation;
switchably coupling, via the matrix switch, the return electrode of the neural interface selected for neural stimulation to the ground;
switchably coupling, via the matrix switch, the first and second working electrodes to the current source; and
floating the return electrode of the neural interface not selected for neural stimulation.

18. The method of claim 17 and further comprising:
selecting neural sensing after neural stimulation;
selecting either the first or the second neural interface for neural sensing;
switchably coupling, via the matrix switch, all the electrodes to the ground for a set period of time greater than 10 microseconds;
switchably coupling, via the matrix switch, the return electrode of the neural interface selected for neural sensing to the ground;
switchably coupling, via the matrix switch, all the first and second working electrodes to the input of the signal processor; and
floating the return electrode of the neural interface not selected for neural sensing.

19. The method of claim 16 and further comprising:
selecting neural sensing;
selecting either the first or the second neural interface for neural sensing;
switchably coupling, via the matrix switch, the return electrode of the neural interface selected for neural sensing to the ground;
switchably coupling, via the matrix switch, all the first and second working electrodes to the input of the signal processor; and
floating the return electrode of the neural interface not selected for neural sensing.

20. The method of claim 16, wherein the first neural interface comprises at least one of a nerve cuff, a helical cuff, paddle electrodes, or an electrode array, and the second neural interface comprises at least one of a nerve cuff, a helical cuff, paddle electrodes, or an electrode array.
